(19) United States
(12) Patent Application Publication
(10) Pub. No.: US 2019/0000853 A1
(43) Pub. Date: Jan. 3, 2019

(54) ANTIVIRAL 4-(2-AMINO-6-HETEROCYLYL-9H-PURIN-9-YL)-2-CYCLOPENTENE-1 -METHANOL COMPOUNDS

(71) Applicant: THE UNIVERSITY OF LIVERPOOL, Liverpool (GB)

(72) Inventors: Paul O'NEILL, Liverpool (GB); Neil BERRY, Liverpool (GB)

(21) Appl. No.: 15/748,955

(22) PCT Filed: Jul. 29, 2016
(86) PCT No.: PCT/GB2016/052356

§ 371 Date: Jan. 30, 2018

(30) Foreign Application Priority Data
Jul. 31, 2015 (GB) 1513601.3

(51) Int. Cl. A61K 31/52; A61P 31/18

(52) U.S. Cl. CPC A61K 31/52; A61P 31/18

(57) ABSTRACT

The present invention relates to certain antiviral compounds of formula I as defined herein that function as nucleoside reverse transcriptase inhibitors. The present invention also relates to processes for the preparation of these compounds, pharmaceutical compositions comprising them and to their use for the treatment of retroviral infections, and in particular their use in the treatment of HIV-1 virus.

### FIELD OF THE INVENTION

The present invention relates to certain antiviral compounds that function by the inhibition of nucleoside reverse transcriptase. The present invention also relates to processes for the preparation of these compounds, to pharmaceutical compositions comprising them and to their use in the treatment of retroviral infections, and in particular their use in the treatment of HIV-1 virus.

### BACKGROUND OF THE INVENTION

Human immunodeficiency virus (HIV) is a virus that attacks the immune system and, as a consequence, weakens the body's ability to fight infection and disease. The HIV virus can be subdivided into two main types, HIV-1 and HIV-2, which differ genetically. Of the two subtypes, HIV-1 is the predominate strain amongst humans.

Abacavir, a nucleoside reverse transcriptase inhibitor, is a drug used as part of a combination therapy for the treatment of HIV-1. It is a guanosine-analogue pro-drug, and once metabolised to carbovir in vivo, it acts as a DNA chain terminator.

One drawback of abacavir treatment is a high frequency of CD8+ T-cell-mediated hypersensitivity reactions in individuals carrying the HLA risk allele B*57:01.

To activate T-cells, abacavir interacts directly with endogenous HLA-B*57:01, altering the repertoire of peptides displayed on the cell surface.

The risk of hypersensitivity reactions in patients carrying the HLA risk allele B*57:01 is sufficiently severe that genetic screening for patients carrying the HLA risk allele B*57:01 needs to be conducted prior to commencing treatment.

There is, therefore, a need for new drug molecules that are effective inhibitors of nucleoside reverse transcriptase and which do not cause adverse hypersensitivity reactions in patients carrying the HLA risk allele B*57:01.

The present invention was devised with the foregoing in mind.

### SUMMARY OF THE INVENTION

In one aspect, the present invention provides a compound as defined herein, or a pharmaceutically acceptable salt or solvate thereof.

In another aspect, the present invention provides a pharmaceutical composition comprising a compound as defined herein, or a pharmaceutically acceptable salt or solvate thereof, in combination with one or more pharmaceutically acceptable excipients.

In another aspect, the present invention provides a compound as defined herein, or a pharmaceutically acceptable salt or solvate thereof, or a pharmaceutical composition as defined herein, for use in therapy.

In another aspect, the present invention provides a compound as defined herein, or a pharmaceutically acceptable salt or solvate thereof, or a pharmaceutical composition as defined herein, for use in the treatment of retroviral infections.

In another aspect, the present invention provides a compound as defined herein, or a pharmaceutically acceptable salt or solvate thereof, or a pharmaceutical composition as defined herein, for use in the treatment of HIV.

In another aspect, the present invention provides a compound as defined herein, or a pharmaceutically acceptable salt or solvate thereof, or a pharmaceutical composition as defined herein, for use in the treatment of HIV-1.

In another aspect, the present invention provides a compound as defined herein, or a pharmaceutically acceptable salt or solvate thereof, or a pharmaceutical composition as defined herein, for use in the treatment of HIV-1 in patients carrying the HLA risk allele B*57:01.

In another aspect, the present invention provides a method of inhibiting nucleoside reverse transcriptase in vitro or in vivo by administering a compound as defined herein, or a pharmaceutically acceptable salt or solvate thereof, or a pharmaceutical composition as defined herein.

In another aspect, the present invention provides a method of treating a retroviral infection in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, the present invention provides a method of treating HIV in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, the present invention provides a method of treating HIV-1 in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, the present invention provides a method of treating HIV-1 in a patient carrying the HLA risk allele B*57:01, said method comprising administering to said patient a therapeutically effective amount of a compound as defined herein, or a pharmaceutically acceptable salt or solvate thereof, or a pharmaceutical composition as defined herein.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkyl-(1-2C)alkylene” means a (3-8C)cycloalkyl group covalently attached to a (1-2C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

“Heterocyclyl(1-2C)alkyl” means a heterocyclyl group covalently attached to a (1-2C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-2C)alkyl” means a heteroaryl group covalently attached to a (1-2C)alkylene group, both of which are defined herein. Examples of heteroaralkyl groups include pyridin-3-ylmethyl, 3-(benzofuran-2-yl)ethyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-2C)alkyl” means an aryl group covalently attached to a (1-2C)alkylene group, both of which are defined herein. Examples of aryl-(1-2C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to compounds, or a pharmaceutically acceptable salts or solvates thereof, having the structural formula (I) shown below:

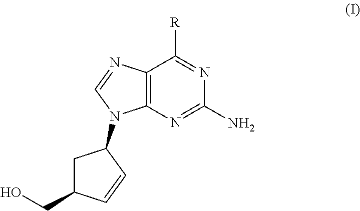

wherein R is a 3 to 6 membered nitrogen-linked heterocyclic ring, and wherein:
(i) the nitrogen-linked heterocyclic ring optionally comprises one or two additional heteroatoms selected from nitrogen, oxygen or sulphur;
(ii) the nitrogen linked heterocyclic ring is optionally linked to a second 3 to 6 membered ring by a spiro carbon atom, the second 3 to 6 membered ring optionally comprising one or two further heteroatoms selected from nitrogen, oxygen or sulphur; and/or
(iii) R is optionally substituted with one or more substituents selected from H, oxo, (1-6C) alkyl, (3-6C) cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, cyano, hydroxyl, amino, nitro, (1-4C)alkoxy, (1-4C)haloalkyl, (1-4C)haloalkoxy;
with the proviso that R is not an unsubstituted azetidin-1-yl ring;
or a pharmaceutically acceptable salt or solvate thereof.

The compounds of the present invention are potent anti-retroviral agents and, compared to abacavir, they are predicted to exhibit a reduced risk of causing hypersensitivity in patients carrying the HLA risk allele B*57:01. The compounds of the present invention are therefore viable alternative agents for the treatment of retroviral infections such as HIV.

Particular compounds of the invention include, for example, compounds of the formula (I), or pharmaceutically acceptable salts and/or solvates thereof, wherein, unless otherwise stated, R, and any associated substituent group has any of the meanings defined hereinbefore or in any of paragraphs (1) to (24) hereinafter:—
(1) R is a 3 to 6 membered nitrogen-linked heterocyclic ring, wherein:
   (i) the nitrogen-linked heterocyclic ring optionally comprises one or two additional heteroatoms selected from nitrogen, oxygen or sulphur;
   (ii) the nitrogen linked heterocyclic ring is optionally linked to a second 3 to 6 membered ring by a spiro carbon atom, the second 4 to 6 membered ring optionally comprising one or two further heteroatoms selected from nitrogen, oxygen or sulphur; and/or
   (iii) R is optionally substituted with one or more substituents selected from H, oxo, (1-6C) alkyl, (3-6C)cycloalkyl, halo, cyano, hydroxyl, amino, nitro, (1-4C) alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, cyano, hydroxyl, amino, nitro, (1-4C)alkoxy, (1-4C)haloalkyl, (1-4C)haloalkoxy;
   with the proviso that R is not an unsubstituted azetidin-1-yl ring;
(2) R is a 3 to 6 membered nitrogen-linked heterocyclic ring, wherein:
   (i) the nitrogen-linked heterocyclic ring optionally comprises one or two additional heteroatoms selected from nitrogen, oxygen or sulphur; and/or
   (ii) R is optionally substituted with one or more substituents selected from H, oxo, (1-6C)alkyl, (3-6C)cycloalkyl, halo, cyano, hydroxyl, amino, nitro, (1-4C)alkoxy, (1-4C)haloalkyl, (1-4C)haloalkoxy, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, hydroxyl, amino, (1-4C)alkoxy or (1-4C)haloalkyl;
   with the proviso that R is not an unsubstituted azetidin-1-yl ring;
(3) R is a 3 to 6 membered nitrogen-linked heterocyclic ring, wherein:
   (i) the nitrogen-linked heterocyclic ring optionally comprises one or two additional heteroatoms selected from nitrogen, oxygen or sulphur; and/or
   (ii) R is optionally substituted with one or more substituents selected from oxo, (1-6C)alkyl, (3-6C)cycloalkyl, halo, cyano, hydroxyl, amino, nitro, (1-4C)alkoxy, (1-4C)haloalkyl, (1-4C)haloalkoxy, wherein any alkyl or cycloalkyl, group is optionally further substituted with one or more substituents selected from (1-4C)alkyl, halo, hydroxyl, amino, (1-4C)alkoxy or (1-4C)haloalkyl;
   with the proviso that R is not an unsubstituted azetidin-1-yl ring;
(4) R is a 3 to 6 membered nitrogen-linked heterocyclic ring, which is optionally substituted with one or more substituents selected from oxo, (1-6C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, cyano, hydroxyl, amino, nitro, (1-4C)alkoxy, (1-4C)haloalkyl, (1-4C)haloalkoxy;
   with the proviso that R is not an unsubstituted azetidin-1-yl ring;
(5) R is a 3 to 6 membered nitrogen-linked heterocyclic ring, which is optionally substituted with one or more substituents selected from oxo, (1-6C)alkyl, (3-6C)cycloalkyl, halo, cyano, hydroxyl, amino, nitro, (1-4C)alkoxy, (1-4C)haloalkyl, (1-4C)haloalkoxy, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, hydroxyl, amino, (1-4C)alkoxy or (1-4C)haloalkyl;
(6) R is a 3 to 6 membered nitrogen-linked heterocyclic ring, which is optionally substituted with one or more substituents selected from oxo, (1-6C)alkyl, (3-6C)cycloalkyl, halo, cyano, hydroxyl, amino, nitro, (1-4C)alkoxy, (1-4C)haloalkyl, (1-4C)haloalkoxy, wherein any alkyl or cycloalkyl, group is optionally further substituted with one or more substituents selected from (1-4C)alkyl, halo, hydroxyl, amino, (1-4C)alkoxy or (1-4C)haloalkyl;
(7) R is a group of the formula:

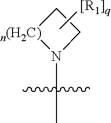

wherein:
[symbol] denotes the point of attachment;
R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
n is an integer selected from 1 to 3; and
q is an integer selected from 0 to 3.
(8) R is a group of the formula:

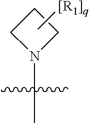

wherein:
[symbol] denotes the point of attachment;
R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy; and
q is an integer selected from 1 to 3.
(9) R is a group of the formula:

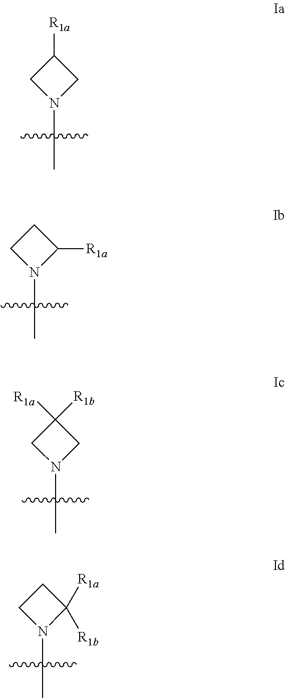

wherein:
[symbol] denotes the point of attachment;
R₁ₐ a substituent selected from (1-4C)alkyl, (3-6C)cycloalkyl, halo, amino, cyano, hydroxyl, nitro, (1-4C)alkoxy, (1-2C)haloalkyl, (1-2C)haloalkoxy, carboxyl, carbamoyl, sulphamoyl or amido, wherein any alkyl or cycloalkyl group is optionally further substituted with one or more substituents selected from (1-2C)alkyl, halo, hydroxyl, amino, (1-2C)alkoxy, (1-2C)haloalkyl; and
R_{1b} a substituent selected from (1-4C)alkyl, halo or hydroxyl;
or R₁ₐ and R_{1b} are optionally linked such that, together with the carbon atom to which they are attached, they form a spiro linked 3 to 6 membered ring.
(10) R is a croup of the formula:

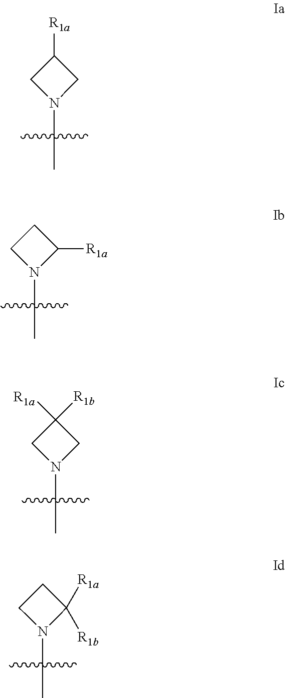

wherein:
[symbol] denotes the point of attachment;
R₁ₐ a substituent selected from (1-4C)alkyl, (3-6C)cycloalkyl, halo, amino, cyano, hydroxyl, nitro, (1-4C)alkoxy, (1-2C)haloalkyl, (1-2C)haloalkoxy, wherein any alkyl or cycloalkyl group is optionally further substituted with one or more substituents selected from (1-2C)alkyl, halo, hydroxyl, amino, (1-2C)alkoxy, (1-2C)haloalkyl; and
R_{1b} a substituent selected from (1-4C)alkyl, halo or hydroxyl;
(11) R is a group of the formula:

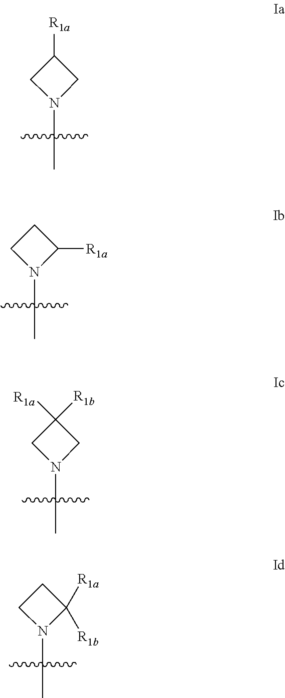

wherein:
[symbol] denotes the point of attachment;
R₁ₐ a substituent selected from (1-4C)alkyl, (3-6C)cycloalkyl, halo, amino, cyano, hydroxyl, (1-4C)alkoxy, (1-2C)haloalkyl, wherein any alkyl group is optionally further substituted with one or more substituents selected from (1-2C)alkyl, halo or hydroxyl; and
R_{1b} a substituent selected from (1-4C)alkyl, halo or hydroxyl;
(12) R is a group of the formula:

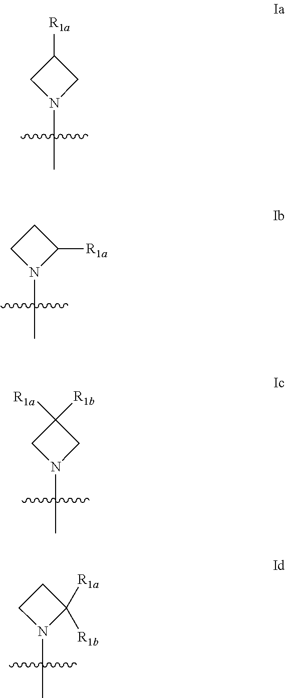

wherein:
[symbol] denotes the point of attachment;
R₁ₐ a substituent selected from (1-4C)alkyl, (3-6C)cycloalkyl, halo, amino, cyano, hydroxyl, (1-4C)alkoxy, (1-2C)haloalkyl, wherein any alkyl group is optionally further substituted with one or more substituents selected from (1-2C)alkyl, halo or hydroxyl; and
R_{1b} a substituent selected from methyl, fluoro or hydroxyl;
(13) R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
(14) R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
(15) R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, wherein any alkyl or cycloalkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
(16) R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, wherein any alkyl or cycloalkyl group is optionally further substituted with one or more substituents selected from (1-2C)alkyl, halo, hydroxyl, amino, (1-2C)alkoxy, (1-2C)haloalkyl;
(17) R₁ is a substituent selected from oxo, (1-4C)alkyl, (3-6C)cycloalkyl, halo, cyano, hydroxyl, amino, (1-4C)alkoxy, (1-4C)haloalkyl, wherein any alkyl group is optionally further substituted with one or more substituents selected from (1-2C)alkyl, halo or hydroxyl;
(18) n is an integer selected from 1 to 3;
(19) n is an integer selected from 1 to 2;
(20) n is 1;
(21) q is an integer selected from 0 to 3.
(22) q is an integer selected from 1 to 3.
(23) q is an integer selected from 1 to 2.
(24) q is 1.

In a particular group of compounds of the invention, the compounds have the structural formula Ia (a sub-definition of formula (I)) shown below:

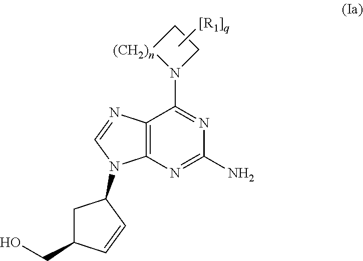

wherein R₁, n and q each have any one of the meanings defined herein; or a pharmaceutically acceptable salt, hydrate and/or solvate thereof.

In an embodiment of the compounds of formula Ia:
R₁ is as defined in any one of paragraphs (13) to (17) above;
n is as defined in any one of paragraphs (18) to (20) above; and
q is as defined in any one of paragraphs (21) to (24) above.

In an embodiment of the compounds of formula Ia:
R₁ is as defined in any one of paragraphs (13) to (17) above;
n is as defined in any one of paragraphs (19) or (20) above; and
q is as defined in any one of paragraphs (23) or (24) above.

In an embodiment of the compounds of formula Ia:
R₁ is as defined in any one of paragraphs (13) to (17) above;
n is as defined in paragraph (20) above; and
q is as defined in paragraph (23) above.

In an embodiment of the compounds of formula Ia:
R₁ is as defined in any one of paragraphs (15) to (17) above;
n is as defined in any one of paragraphs (19) or (20) above; and
q is as defined in any one of paragraphs (23) or (24) above.

In an embodiment of the compounds of formula Ia:
R₁ is as defined in any one of paragraphs (15) to (17) above;
n is as defined in paragraph (20) above; and
q is as defined in paragraph (24) above.

Particular compounds of the present invention include any one of the following compounds, or a pharmaceutically acceptable salt or solvate thereof:

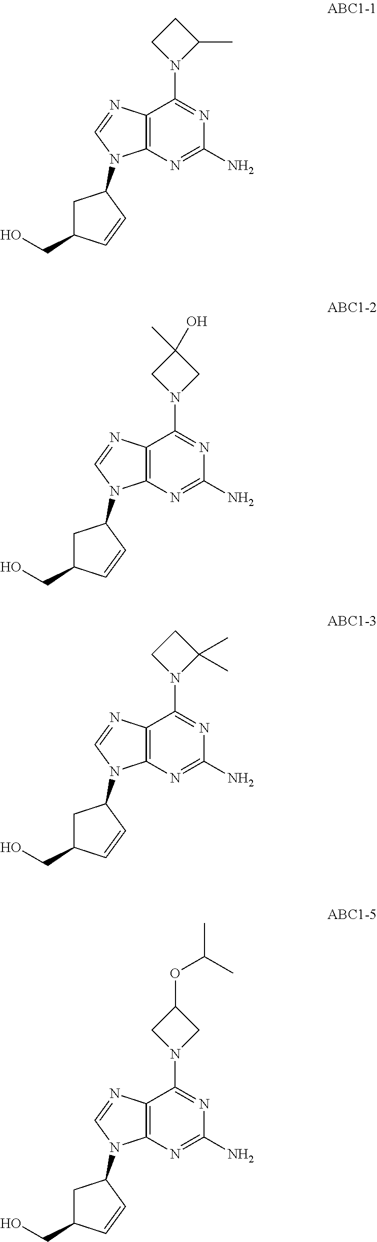 [SMILES: CC1CCN1c1nc(N)nc2c1ncn2[C@H]1C=C[C@@H](CO)C1 ; CC1(O)CN(c2nc(N)nc3c2ncn3[C@H]2C=C[C@@H](CO)C2)C1 ; CC1(C)CCN1c1nc(N)nc2c1ncn2[C@H]1C=C[C@@H](CO)C1 ; CC(C)OC1CN(c2nc(N)nc3c2ncn3[C@H]2C=C[C@@H](CO)C2)C1]

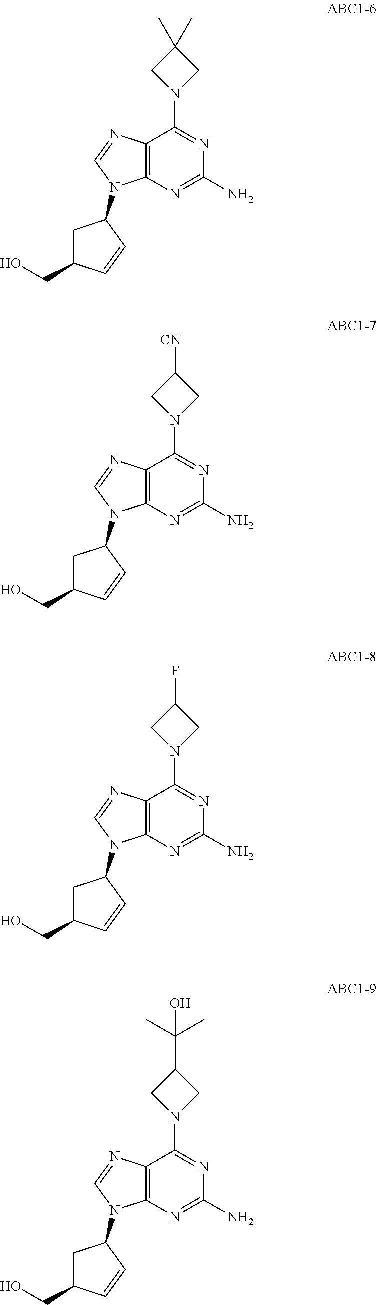

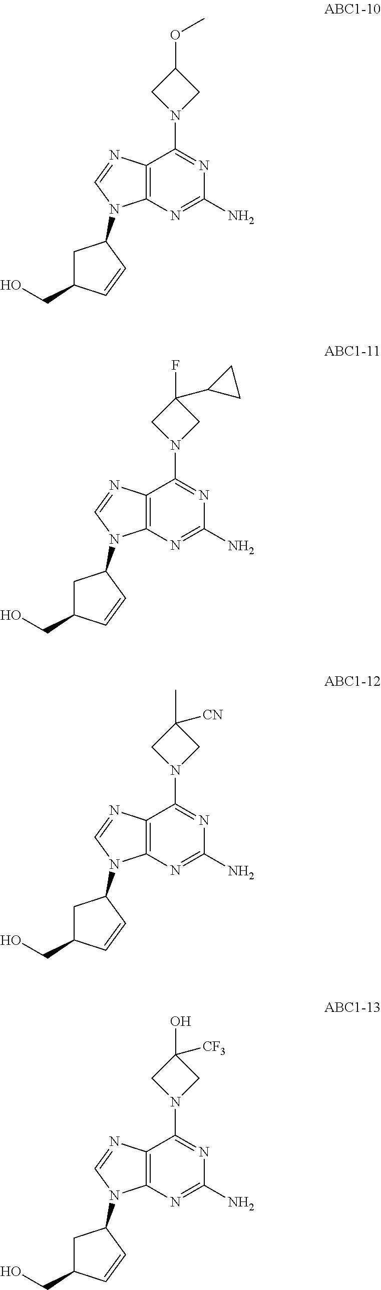 [SMILES: COC1CN(c2nc(N)nc3c2ncn3[C@H]2C=C[C@@H](CO)C2)C1 ; Nc1nc(N2CC(F)(C3CC3)C2)c2ncn([C@H]3C=C[C@@H](CO)C3)c2n1 ; CC1(C#N)CN(c2nc(N)nc3c2ncn3[C@H]2C=C[C@@H](CO)C2)C1 ; Nc1nc(N2CC(O)(C(F)(F)F)C2)c2ncn([C@H]3C=C[C@@H](CO)C3)c2n1]

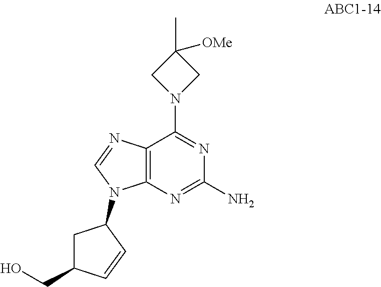 [SMILES: COC1(C)CN(c2nc(N)nc3c2ncn3[C@H]2C=C[C@@H](CO)C2)C1]

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650, or less than 600. More preferably, the molecular weight is less than 550 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiviral activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the formula (I) may exist in solvated as well as unsolvated forms such as, for example, hydrated forms. It is to be understood that the invention encompasses all such solvated forms that possess antiviral activity.

It is also to be understood that certain compounds of the formula I may exhibit polymorphism, and that the invention encompasses all such forms that possess antiviral activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

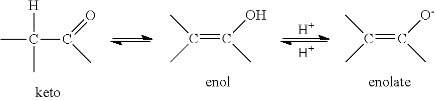

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula (I) as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C1-6alkyl esters such as methyl, ethyl and tert-butyl, C1-6alkoxymethyl esters such as methoxymethyl esters, C1-6alkanoyloxymethyl esters such as pivaloyloxymethyl esters, 3-phthalidyl esters, C3-8cycloalkylcarbonyloxy-C1-6alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C1-6alkoxycarbonyloxy-C1-6alkyl esters such as methoxycarbonyloxymethyl and 1-methoxycarbonyloxyethyl esters.

A suitable pharmaceutically acceptable pro-drug of a compound of the Formula (I) that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C1-10alkanoyl groups such as acetyl, benzoyl, phenylacetyl and substituted benzoyl and phenylacetyl groups, C1-10alkoxycarbonyl groups such as ethoxycarbonyl, N,N—(C1-6)2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C1-4alkylamine such as methylamine, a (C1-4alkyl)2amine such as dimethylamine, N-ethyl-N-methylamine or diethylamine, a C1-4alkoxy-C2-4alkylamine such as 2-methoxyethylamine, a phenyl-C1-4alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C1-10alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

Once a compound of formula (I) has been synthesised by any one of the processes defined herein, the processes may then further comprise the additional steps of:

(i) removing any protecting groups present;
(ii) converting the compound formula (I) into another compound of formula (I);
(iii) forming a pharmaceutically acceptable salt, hydrate or solvate thereof; and/or
(iv) forming a prodrug thereof.

The resultant compounds of formula (I) can be isolated and purified using techniques well known in the art.

Suitably, a compound of the present invention is prepared by:

(a) reacting a compound of formula (A):

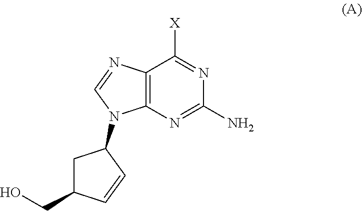

(wherein X is a suitable leaving group (e.g. halo));

with a compound of formula (B):

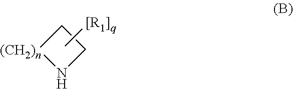

(wherein R₁, n and q are each as defined herein);

in the presence of a suitable base (e.g. DIPEA); and

(b) optionally thereafter, and if necessary:
i) removing any protecting groups present;
ii) converting the compound formula I into another compound of formula I; and/or
iii) forming a pharmaceutically acceptable salt or solvate thereof.

A person of skill in the art will be able to select suitable reaction conditions (e.g. temperature, pressures, reaction times, concentration etc.) for such a synthesis.

Suitably, the reaction between compounds of formula A and formula B takes place in the presence of a suitable solvent. Any suitable solvent or solvent mixture may be used for this reaction. A non-limiting list of suitable anhydrous solvents includes methanol, ethanol, isopropanol, DMF, DMSO and CHCl₃.

Suitably, the reactions between compounds of formula A and formula B proceeds at elevated temperatures. More suitably, the reaction proceeds at temperatures between 25 and −100° C. Most suitably, the reaction proceeds at temperatures between 50° C. and 80° C.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent an antiviral condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Modelling

As a means of reliably predicting both the adopted binding poses and activity towards T-Cells displayed by the compounds of the present invention, various modelling (docketing) protocols may be utilised. In particular, a docking protocol reproducing the native crystal structure of abacavir in the HLA-B*57:01 F-pocket binding site may be utilised to predict binding affinity and poses, as detailed hereinbelow (Nature, 486, 554-558 (2012)). This protocol allows for binding poses adopted by the compounds of the present invention to be predicted, along with predicted binding strengths, giving a GoldScore value of the interaction. As previously discovered, a definitive relationship between GoldScore value and T-Cell response may be established which, in turn, makes the GoldScore value a reliable predictor of T Cell activity (paper submitted to AIDS (2015), Towards Depersonalised Abacavir Therapy: Chemical Modification Eliminates HLA-B*57:01-Restricted CD8+ T-Cell Activation)

In a particular embodiment, the compounds of the present invention have a GoldScore value equal to or less than 75. Suitably, the compounds of the present invention have a GoldScore value equal to or less than 70. More suitably, the compounds of the present invention have a GoldScore value equal to or less than 65. Most suitably, the compounds of the present invention have a GoldScore value equal to or less than 63.

### Therapeutic Uses and Applications

The present invention provides compounds, or pharmaceutically acceptable salts, hydrates or solvates thereof, which function as inhibitors of nucleoside reverse transcriptase.

The present invention therefore provides a method of inhibiting nucleoside reverse transcriptase activity in vitro or in vivo, said method comprising contacting a cell with an effective amount of a compound, or a pharmaceutically acceptable salt, hydrate or solvate thereof, as defined herein.

The present invention also provides a method of treating a disease or disorder in which nucleoside reverse transcriptase activity is implicated in a patient in need of such treatment, said method comprising administering to said patient a therapeutically effective amount of a compound, or a pharmaceutically acceptable salt, hydrate or solvate thereof, or a pharmaceutical composition as defined herein.

The present invention also provides a method of selectively inhibiting nucleoside reverse transcriptase over CD8+ T-cells, activation of which results in hypersensitivity, both in vitro and in vivo, said method comprising contacting a cell with an effective amount of a compound, or a pharmaceutically acceptable salt, hydrate or solvate thereof, as defined herein.

The present invention provides a method of treating human immunodeficiency virus (HIV) in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of treating HIV-1 in a patient in need of such treatment, said method comprising administering to said patient a therapeutically effective amount of a compound, or a pharmaceutically acceptable salt, hydrate or solvate thereof, or a pharmaceutical composition as defined herein.

The present invention also provides a method of treating HIV-1 in a patient carrying the HLA risk allele B*57:01 in need of such treatment, said method comprising administering to said patient a therapeutically effective amount of a compound, or a pharmaceutically acceptable salt, hydrate or solvate thereof, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use in the treatment of HIV. In a particular embodiment, the HIV is HIV-1.

The present invention provides a compound or formula I as defined herein, or a pharmaceutically acceptable salt, hydrate or solvate thereof, and including compounds in which R is azetidin-1yl, or a pharmaceutical composition as defined herein (including pharmaceutical compositions comprising a compound in which R is azetidin-1yl) for use in the treatment of HIV-1 in patients carrying the HLA risk allele B*57:01.

Particular compounds of formula I include those defined hereinbefore, including the definitions of R provided in numbered paragraphs (1) to (12), with the exception that compounds in which R is azetidin-1-yl are included.

The present invention provides a use of a compound, or a pharmaceutically acceptable salt, hydrate or solvate thereof, as defined herein in the manufacture of a medicament for the treatment of a HIV. Suitably, the HIV is HIV-1.

The present invention provides a use of a compound or formula I as defined herein, or a pharmaceutically acceptable salt, hydrate or solvate thereof, and including compounds in which R is azetidin-1yl, or a pharmaceutical composition as defined herein (including pharmaceutical compositions comprising a compound in which R is azetidin-1yl), in the manufacture of a medicament for the treatment of HIV-1 in patients carrying the HLA risk allele B*57:01.

### Examples

### Materials and Methods

### Chemical Analyses

All ¹H and ¹³C NMR were run on a Bruker ARX 400 (400 MHz) nuclear magnetic resonance spectrometer and all deuterated solvents used are as stated within the Online Repository. Tetramethylsilane was used as the internal reference. Multiplicities are recorded as singlet (s); doublet (d); triplet (t); multiplet (m) and broad (br). All J couplings are recorded in Hertz. Mass spectra were recorded on a Micromass LCT Mass Spectrometer. Accurate mass values are stated within ±5 ppm. Infrared spectrometry was measured using a Jasco FT/IR-4200 spectrometer and samples were run neat. Melting points were determined.

### Chemical Synthesis

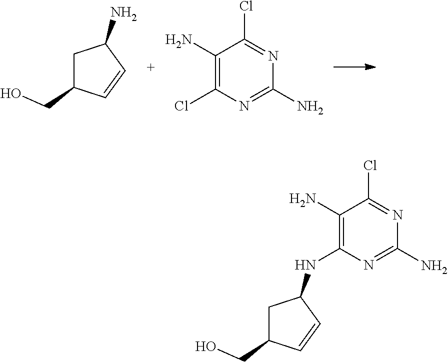 [SMILES: N[C@H]1C=C[C@@H](CO)C1 ; Nc1nc(Cl)c(N)c(Cl)n1 ; Nc1nc(Cl)c(N)c(N[C@H]2C=C[C@@H](CO)C2)n1]

### ((1S,4R)-4-((2,5-Diamino-6-chloropyrimidin-4-yl)amino)cyclopent-2-en-1-yl)methanol

To (1S,4R)-4-amino-2-cyclopentene-1-methanol tartrate salt (1 g, 4 mmol) in n-BuOH (10 mL) was added 2,5-diamino-4,6-dichloropyrimidine (DADCP, 0.69 g, 4 mmol) and NaHCO₃ (1.12 g, 13 mmol) and the mixture was allowed to stir for 16 hr at 95° C. The mixture was cooled and filtered under vacuum. The resulting solvent from the filtrate was removed in vacuo. The crude product was purified by column chromatography (1:49 to 1:24 MeOH-DCM) to yield the title compound (0.52 g, 53%) as a pale brown solid. m.p.: 157-159° C. (lit. m.p. 158.5-160.5° C.) (29). ¹H NMR (MeOD): δ 5.87 (ddd, J=5.5, 2.0, 1.9 Hz, 1H), 5.80 (ddd, J=5.6, 2.0, 2.0 Hz, 1H), 5.07-5.15 (m, 1H), 3.47-3.56 (m, 1H), 3.28 (dt, J=3.2, 1.6 Hz, 1H), 2.79-2.83 (m, 1H), 2.53 (ddd, J=13.4, 6.4, 6.4 Hz, 1H), 1.36 (ddd, J=13.4, 6.3, 5.9 Hz, 1H). ¹³C NMR (MeOD): δ 158.2, 157.3, 143.2, 135.8, 133.9, 114.4, 66.4, 57.8, 57.7, 35.8. m/z (ES+) 256.0955 calculated for C₁₀H₁₅N₅O³⁵Cl: [M+H]⁺ 256.0965 and 258.0928 calculated for C₁₀H₁₅N₅O³⁷Cl: [M+H]⁺ 258.0936. IR (cm⁻¹): 3451 (N—H), 3324 (N—H), 3125 (br, O—H), 2931 (C—H), 1570 (C═C aromatic), 1430 (CH₂), 690 (C—Cl).

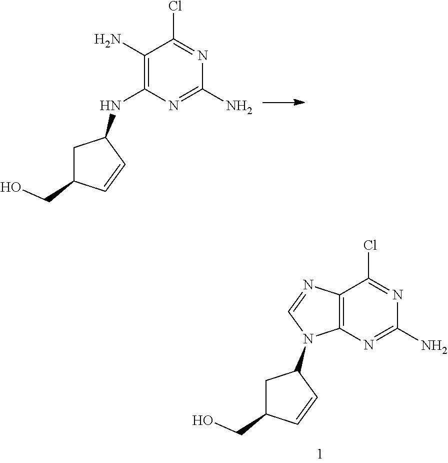

### ((1S,4R)-4-(2-Amino-6-chloro-9H-purin-9-yl)cyclopent-2-en-1-yl)methanol (1)

To a solution of ((1S,4R)-4-((2,5-Diamino-6-chloropyrimidin-4-yl)amino)cyclopent-2-en-1-yl)methanol (0.5 g, 2 mmol) in n-BuOH (15 mL) was added TEOF (0.4 mL, 2.4 mmol) and H₂SO₄ (0.05 μL, 0.1 mmol). The brown solution was allowed to stir for 20 hr at 70° C. The solution was allowed to cool whereupon the solvent was removed in vacuo and the crude mixture was purified by column chromatography (1:99 to 5:95 MeOH-DCM) to yield the title compound 1 (0.45 g, 87%) as a brown solid. m.p.: 158-161° C. (lit. m.p. 160-162° C.) (30). ¹H NMR (CDCl₃): δ 7.95 (s, 1H), 6.18 (ddd, J=5.5, 2.2, 2.1 Hz, 1H), 5.82 (ddd, J=5.5, 2.2, 2.0 Hz, 1H), 5.52-5.56 (m, 1H), 3.85 (dd, J=10.7, 4.2 Hz, 1H), 3.76 (dd, J=10.7, 3.9 Hz, 1H), 3.48 (MeOH), 3.09-3.13 (m, 1H), 2.81 (ddd, J=14.4, 6.4, 6.2 Hz, 1H), 1.98 (ddd, J=14.4, 6.2, 5.3 Hz, 1H). ¹³C NMR (CDCl₃): δ 159.1, 153.5, 151.6, 142.2, 139.5, 130.0, 115.9, 65.0, 61.1, 48.1, 33.6. m/z (ES+) 288.0626 calculated for C₁₁H₁₂N₅O²³Na³⁵Cl: [M+Na]⁺ 288.0628 and 290.0603 calculated for C₁₁H₁₂N₅O²³Na³⁷Cl: [M+Na]⁺ 290.0599. IR (cm⁻¹): 3325 (N—H), 3205 (N—H), 1581 (C═C aromatic), 1246 (C—O), 640 (C—Cl).

### General Procedure for Preparation of Compounds ABC1-1 to ABC1-10

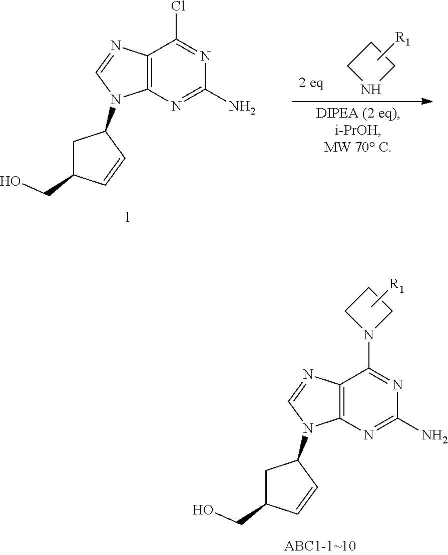

Compound 1 (150.00 mg, 564.55 umol, 1.00 eq), azetidine (2.00 eq) and N,N-diisopropylethylamine (145.92 mg, 1.13 mmol, 2.00 eq) were taken up into a microwave tube in isopropyl alcohol (2.00 mL). The sealed tube was heated at 70° C. for 2 hours under microwave. LCMS showed that the starting material was consumed completely. The mixture was concentrated in vacuum to give crude product.

### ABC1-1

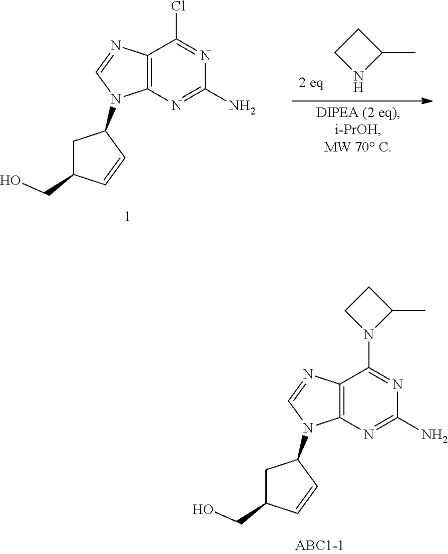

The crude product was purified by preparative TLC purification to give ABC1-1 (54.8 mg, 182.4 μmol, 32% yield) as a white solid.

LCMS: (M+H⁺): 301.1

¹H NMR: 400 MHz MeOD δ 7.70 (s, 1H), 6.15 (t, J=3.6 Hz, 1H), 5.88 (t, J=2.4 Hz, 1H), 5.47-5.51 (m, 1H), 4.80 (s, 1H), 4.35 (s, 1H), 4.21 (s, 1H), 3.61-3.65 (m, 2H), 2.98 (s, 1H), 2.76-2.98 (m, 1H), 2.73-2.75 (m, 1H), 1.68-2.02 (m, 1H), 1.65-1.67 (m, 1H), 1.57 (d, J=6.4 Hz, 3H).

### ABC1-2

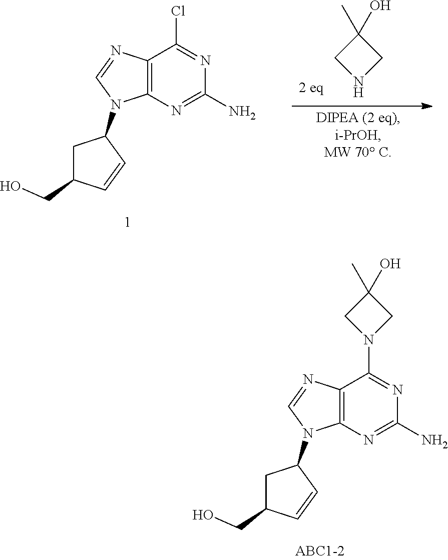

The crude product was purified by preparative TLC purification to give ABC1-2 (104.8 mg, 331.3 μmol, 58% yield) as a white solid.

LCMS: (M+H⁺): 317.1

¹H NMR: 400 MHz MeOD δ 7.73 (s, 1H), 6.15 (t, J=3.6 Hz, 1H), 5.88 (t, J=2.4 Hz, 1H), 5.48-5.49 (m, 1H), 4.20 (s, 1H), 3.57-3.65 (m, 2H), 3.54 (s, 2H), 2.98 (s, 1H), 2.73-2.76 (m, 1H), 1.65-1.70 (m, 1H), 1.63 (d, J=5.6 Hz, 3H).

### ABC1-3

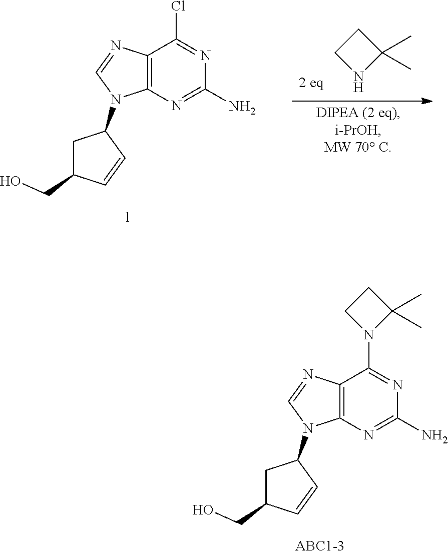

The crude product was purified by preparative TLC purification to give ABC1-3 (109.5 mg, 348.3 μmol, 62% yield) as a white solid.

LCMS: (M+H⁺): 315.2

¹H NMR: 400 MHz MeOD δ 7.64 (s, 1H), 6.12-6.15 (m, 1H), 5.86 (t, J=2.4 Hz, 1H), 5.45-5.47 (m, 1H), 4.38 (s, 1H), 3.56-4.00 (m, 2H), 2.96-2.97 (m, 1H), 2.72-2.75 (m, 1H), 2.18-2.21 (m, 1H), 1.62-1.68 (m, 6H).

### ABC1-4

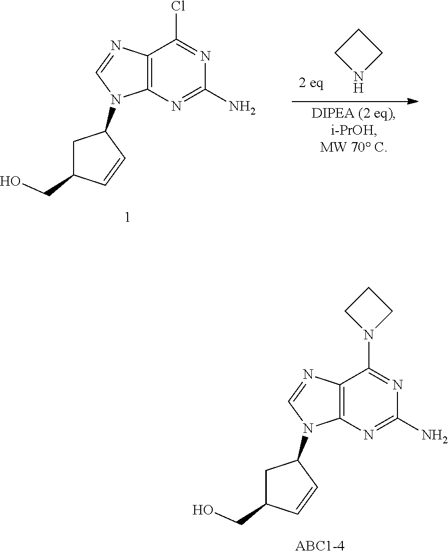

The crude product was purified by preparative TLC purification to give ABC1-4 (57.3 mg, 200.1 μmol, 35% yield) as a white solid.

LCMS: (M+H⁺): 287.1

¹H NMR: 400 MHz MeOD δ 7.71 (s, 1H), 6.14-6.16 (m, 1H), 5.86-5.88 (m, 1H), 5.47-5.50 (m, 1H), 4.80 (s, 1H), 3.56-3.65 (m, 2H), 2.98 (s, 1H), 2.72-2.76 (m, 1H), 2.44-2.47 (m, 2H), 1.63-1.69 (m, 1H)

### ABC1-5

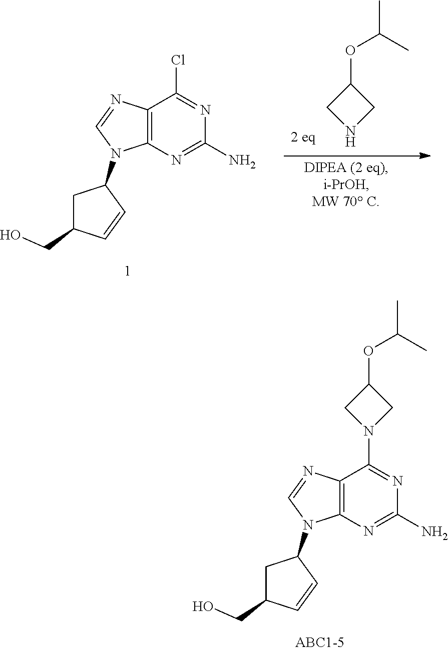

The crude product was purified by preparative TLC purification to give ABC1-5 (126.1 mg, 366.1 μmol, 65% yield) as a white solid.

LCMS: (M+H⁺): 301.2

¹H NMR: 400 MHz MeOD δ 7.73 (s, 1H), 6.14-6.16 (m, 1H), 5.86-5.88 (m, 1H), 5.47-5.51 (m, 1H), 4.57 (s, 2H), 4.14 (s, 1H), 3.61-3.71 (m, 3H), 2.98-3.32 (m, 1H), 2.72-2.98 (m, 1H), 1.64-1.70 (m, 1H), 1.16 (d, J=6.0 Hz, 6H).

### ABC1-6

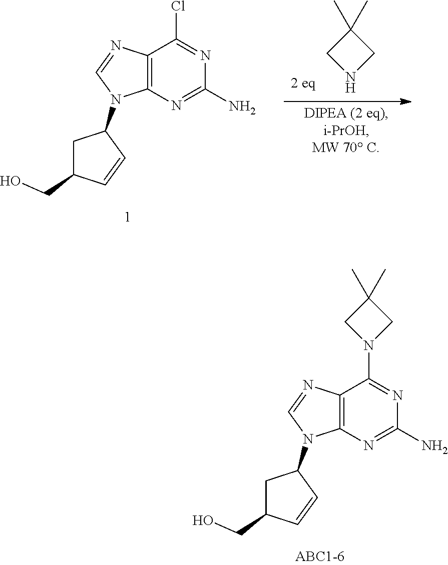

The crude product was purified by preparative TLC purification to give ABC1-6 (71.0 mg, 225.8 μmol, 40% yield) as a yellow solid.

LCMS: (M+H⁺): 315.2

¹H NMR: 400 MHz MeOD δ 7.71 (s, 1H), 6.14-6.16 (m, 1H), 5.86-5.88 (m, 1H), 5.47-5.51 (m, 1H), 4.03 (s, 1H), 3.57-3.65 (m, 2H), 2.97-2.98 (m, 1H), 2.72-2.76 (m, 1H), 1.64-1.70 (m, 1H), 1.35 (s, 6H).

### ABC1-7

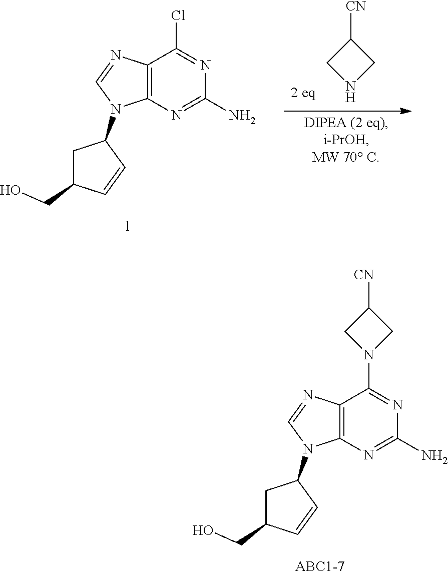

The crude product was purified by preparative TLC purification to give ABC1-7 (79.9 mg, 256.6 μmol, 45% yield) as a white solid.

LCMS: (M+H⁺): 312.2

¹H NMR: 400 MHz MeOD δ 7.63 (s, 1H), 6.08-6.09 (m, 1H), 6.02 (s, 2H), 5.82-5.83 (m, 1H), 5.34-5.37 (m, 1H), 4.68-4.71 (m, 1H), 4.48-4.52 (m, 2H), 4.32-4.33 (m, 1H), 3.88-3.92 (m, 1H), 3.40 (t, J=5.6 Hz, 2H), 2.56-2.83 (m, 1H), 2.42-2.54 (m, 1H), 1.50-1.55 (m, 1H).

### ABC1-8

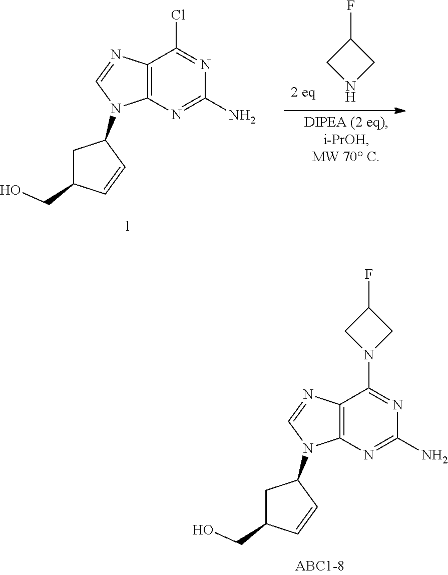 [SMILES: Nc1nc(Cl)c2ncn([C@H]3C=C[C@@H](CO)C3)c2n1 ; FC1CNC1 ; Nc1nc(N2CC(F)C2)c2ncn([C@H]3C=C[C@@H](CO)C3)c2n1]

The crude product was purified by preparative TLC purification to give ABC1-8 (65.0 mg, 213.6 μmol, 38% yield) as a white solid.

LCMS: (M+H⁺): 305.2

¹H NMR: 400 MHz MeOD δ 7.75 (s, 1H), 6.14-6.16 (m, 1H), 5.87-5.88 (m, 1H), 5.50-5.86 (m, 2H), 4.65 (s, 2H), 4.36-4.42 (m, 2H), 3.56-3.65 (m, 2H), 3.28-3.54 (m, 1H), 2.73-2.98 (m, 1H), 1.64-1.71 (m, 1H).

### ABC1-9

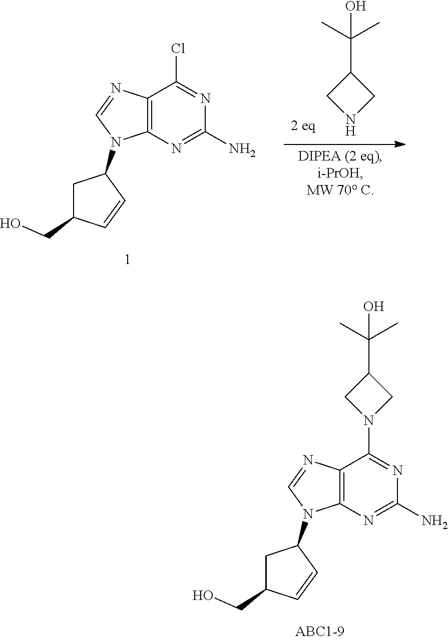

The crude product was purified by preparative TLC purification to give ABC1-9 (134.5 mg, 390.2 μmol, 69% yield) as a white solid.

LCMS: (M+H⁺): 345.2

¹H NMR: 400 MHz MeOD δ 7.73 (s, 1H), 6.15 (s, 1H), 5.87 (s, 1H), 5.50 (s, 1H), 3.57-3.65 (m, 2H), 3.32 (s, 1H), 2.98 (s, 1H), 2.83-2.87 (m, 1H), 2.72-2.76 (m, 1H), 1.65-1.68 (m, 1H), 1.28-1.34 (m, 1H), 1.17 (s, 6H).

### ABC1-10

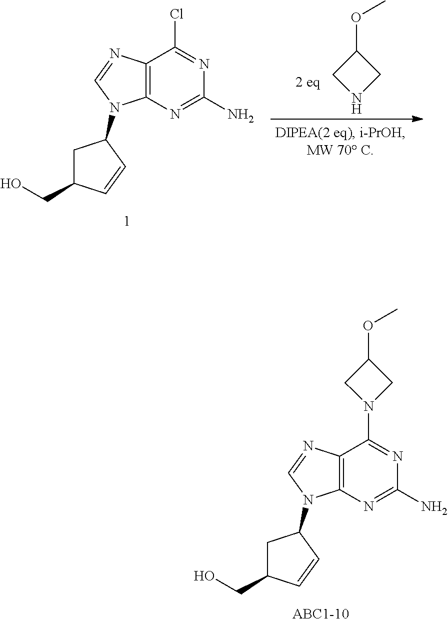

The crude product was purified by preparative TLC purification to give ABC1-10 (83.4 mg, 263.6 μmol, 47% yield) as a white solid.

LCMS: (M+H⁺): 317.2

¹H NMR: 400 MHz MeOD δ 7.73 (s, 1H), 6.15 (t, J=3.6 Hz, 1H), 5.88 (t, J=2.0 Hz, 1H), 5.50 (t, J=5.6 Hz, 1H), 4.53 (s, 1H), 4.37-4.39 (s, 1H), 4.36 (s, 1H), 3.57-3.65 (m, 2H), 3.55 (s, 3H), 2.98 (s, 1H), 2.73-2.78 (m, 1H), 1.63-1.70 (m, 1H).

### Procedure for Preparation of Compounds ABC1-11 to ABC1-14

### ABC1-11

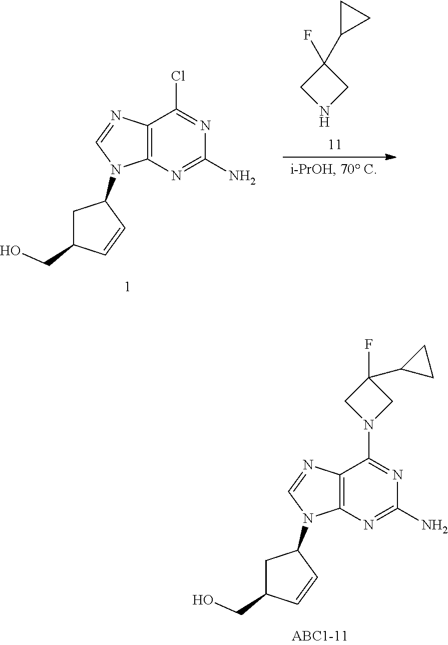 [SMILES: Nc1nc(Cl)c2ncn([C@H]3C=C[C@@H](CO)C3)c2n1 ; FC1(C2CC2)CNC1 ; Nc1nc(N2CC(F)(C3CC3)C2)c2ncn([C@H]3C=C[C@@H](CO)C3)c2n1]

Compound 1 (135 mg, 447 μmol, 1.0 eq), compound 11 (135 mg, 894 μmol, 2.0 eq) and DIPEA (289 mg, 2.2 mmol, 5.0 eq) were dissolved in i-PrOH (3 mL). The solution was heated at 70° C. under microwave for 2 hr. LC-MS showed the reaction was completed. The reaction solution was concentrated and the residue was dissolved in EtOAc (20 mL). The organic phase was washed with water (20 mL). The water phase was extracted with EtOAc (3×10 mL). The combined organic layers were washed with brine (10 mL), dried over Na₂SO₄, filtrated and concentrated to give ABC1-11 (100 mg, 290 μmol, 65% yield) as an off white solid.

¹H NMR: ET5353-5-P1A 400 MHz MeOD δ 7.78 (s, 1H), 6.17-6.18 (m, 1H), 5.89-5.91 (m, 1H), 5.51-5.55 (m, 1H), 4.33 (d, J=16 Hz, 4H), 3.57-3.68 (m, 2H), 3.00-3.01 (m, 1H), 2.75-2.81 (m, 1H), 1.67-1.73 (m, 1H), 1.41-1.45 (m, 1H), 0.65-0.70 (m, 2H), 0.50-0.53 (m, 2H).

LCMS: (M+H)±: 345.2

SFC: 100%

### ABC1-12

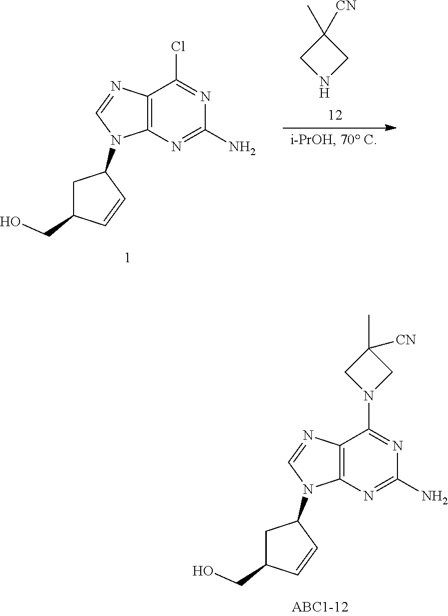

Compound 1 (135 mg, 447 μmol, 1.0 eq), compound 12 (118 mg, 894 μmol, 2.0 eq) and DIPEA (289 mg, 2.2 mmol, 5.0 eq) were dissolved in i-PrOH (3.0 mL). The solution was heated at 70° C. under microwave for 2 hr. LC-MS showed the reaction was completed. The reaction solution was concentrated and the residue was dissolved in EtOAc (20 mL). The organic phase was washed with water (20 mL). The water phase was extracted with EtOAc (3×10 mL). The combined organic layers were washed with brine (10 mL), dried over Na₂SO₄, filtrated and concentrated to give the crude product. The crude product was purified by prep-TLC (Dichloromethane:Methanol=10:1) to give ABC1-12 (75 mg, 231 μmol, 52% yield) as a white solid.

¹H NMR: 400 MHz MeOD δ 7.79 (s, 1H), 6.17-6.19 (m, 1H), 5.89-5.91 (m, 1H), 5.52-5.55 (m, 1H), 4.65 (d, J=8 Hz, 2H), 4.29 (d, J=8 Hz, 2H), 3.57-3.68 (m, 2H), 3.00 (s, 1H), 2.73-2.81 (m, 1H), 1.74 (s, 3H), 1.68-1.73 (m, 1H).

LCMS: (M+H)±: 326.2

SFC: 100%

### ABC1-13

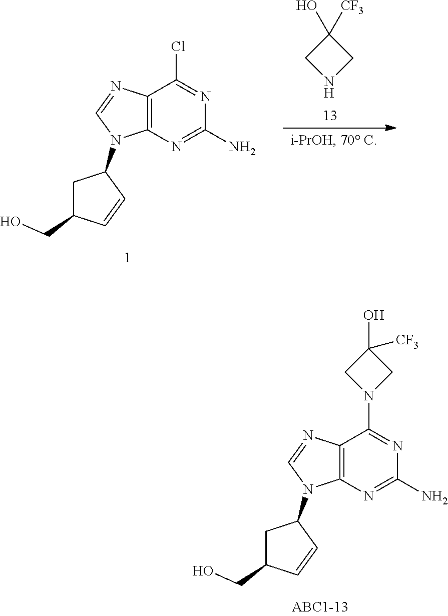 [SMILES: Nc1nc(Cl)c2ncn([C@H]3C=C[C@@H](CO)C3)c2n1 ; OC1(C(F)(F)F)CNC1 ; Nc1nc(N2CC(O)(C(F)(F)F)C2)c2ncn([C@H]3C=C[C@@H](CO)C3)c2n1]

Compound 1 (135 mg, 447 μmol, 1.0 eq), compound 13 (159 mg, 894 μmol, 2.0 eq) and DIPEA (289 mg, 2.2 mmol, 5.0 eq) were dissolved in i-PrOH (3 mL). The solution was heated at 70° C. under microwave for 2 hr. LC-MS showed the reaction was completed. The reaction solution was concentrated and the residue was dissolved in EtOAc (20 mL). The organic phase was washed with water (20 mL). The water phase was extracted with EtOAc (10 mL*3). The combined organic layers were washed with brine (10 mL), dried over Na₂SO₄, filtrated and concentrated to give the crude product. The crude product was purified by prep-TLC (Dichloromethane:Methanol=10:1) to give ABC1-13 (65 mg, 176 μmol, 39% yield) as a white solid.

¹H NMR: 400 MHz MeOD δ 7.79 (s, 1H), 6.17-6.19 (m, 1H), 5.90-5.91 (m, 1H), 5.50-5.54 (m, 1H), 4.60 (s, 2H), 4.29 (d, 2H), 3.57-3.68 (m, 2H), 3.00 (s, 1H), 2.73-2.81 (m, 1H), 1.67-1.74 (m, 1H).

LCMS: (M+H)±: 371.2

SFC: 100%

### ABC1-14

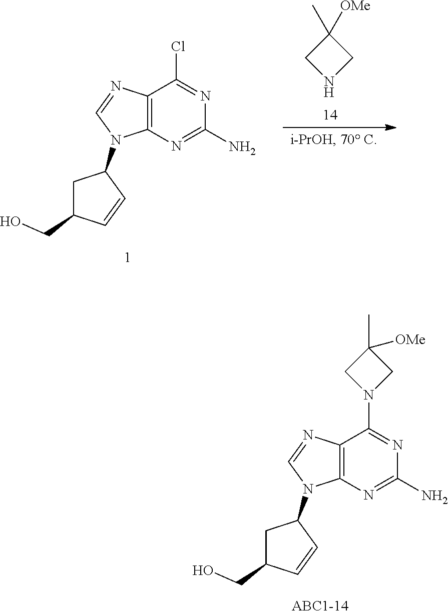

Compound 1 (135 mg, 447 μmol, 1.0 eq), compound 14 (123 mg, 894 μmol, 2.0 eq) and DIPEA (289 mg, 2.2 mmol, 5.0 eq) were dissolved in i-PrOH (3 mL) in a microwave tube. The solution was heated at 70° C. under microwave for 2 hr. LC-MS showed the reaction was completed. The reaction solution was concentrated and the residue was dissolved in EtOAc (20 mL). The organic phase was washed with water (20 mL). The water phase was extracted with EtOAc (10 mL*3). The combined organic layers were washed with brine (10 mL), dried over Na₂SO₄, filtrated and concentrated to give the crude product. The crude product was purified by prep-TLC (Dichloromethane:Methanol=10:1) to give ABC1-14 (65 mg, 197 μmol, 44% yield) as a white solid.

¹H NMR: 400 MHz MeOD δ 7.76 (s, 1H), 6.17-6.19 (m, 1H), 5.91-5.94 (m, 1H), 5.22 (s, 1H), 4.28 (s, 2H), 4.16 (s, 2H), 3.57-3.68 (m, 2H), 3.32 (s, 3H), 3.00 (s, 1H), 2.75-2.81 (m, 1H), 1.66-1.73 (m, 1H), 1.55 (s, 3H).

LCMS: (M+H)±: 331.2

SFC: 100%

### Modelling Studies

### Goldscore Docking—General Procedure

For docking studies, GOLD 5.1 (CCDC Software Limited, Cambridge, UK) was used to find potential binding poses of abacavir analouges in the F-pocket of HLA-B*57:01 (PDB code 3UPR). Hydrogen molecules were added to the protein and the native ligand abacavir was used to define the binding site as residues within 6 Å of the ligand. A scaffold match constraint was used constrain the geometry of the core of each ligand to the binding pose of the core of the abacavir. Aside from this constraint a flexible docking of the R groups was performed. All other settings were left at default. Docking scores ≤63 indicating that activation of T-cells was not predicted to occur.

Calculated docking (or Goldscore) values are shown in Table 1.

TABLE 1
The average Goldscore values for
compounds ABC1-1 to ABC1-14.

| | Average |
|---|---|
| Compound | Goldscore |
| ABC1-1 | 47 |
| ABC1-2 | 55 |
| ABC1-3 | 40 |
| ABC1-4 | 53 |
| ABC1-5 | 54 |
| ABC1-6 | 56 |
| ABC1-7 | 60 |
| ABC1-8 | 52 |
| ABC1-9 | 35 |
| ABC1-10 | 60 |
| ABC1-11 | 58 |
| ABC1-12 | 60 |
| ABC1-13 | 56 |
| ABC1-14 | 64 |

### QSAR Modelling—General Procedure

QSAR models were developed and validated in Pipeline Pilot (BioVia, US) and then applied to the compounds presented above. The model were built using Random Forests approach using the following descriptors: ALogP, Molecular_Weight, Num_H_Donors, Num_H_Acceptors, Num_RotatableBonds, Num_Atoms, Num_Rings, Num_AromaticRings, Molecular_Solubility, Molecular_SurfaceArea, Molecular_PolarSurfaceArea, ECFP4, FCFP4 and MDL keys. All settings were left as default. The model developed showed very good statistics (ROC score (out-of-bag data): 0.7024) and this was applied to the compounds shown above with the results given in Table 2 below. There is a high degree of agreement between the structure-(Goldscore model) and ligand-based approach (QSAR model).

TABLE 2
The predicted T Cell activation
for compounds ABC1-1 to ABC1-14 following QSAR modelling.

| | Predicted T Cell |
|---|---|
| Compound | activation |
| ABC1-1 | Yes |
| ABC1-2 | No |
| ABC1-3 | No |
| ABC1-4 | No |
| ABC1-5 | No |
| ABC1-6 | No |
| ABC1-7 | No |
| ABC1-8 | No |
| ABC1-9 | No |
| ABC1-10 | No |
| ABC1-11 | No |
| ABC1-12 | No |
| ABC1-13 | No |
| ABC1-14 | No |

Antiviral data for compound ABC1-4 is detailed in Antmicrob. Agents Chemother, 1997, 41, 1082, and demonstrates ABC1-4 as being equipotent to Abacavir.

While specific embodiments of the invention have been described herein for the purpose of reference and illustration, various modifications will be apparent to a person skilled in the art without departing from the scope of the invention as defined by the appended claims.

1. A compound of the formula (I), shown below: 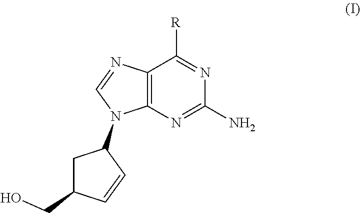
wherein R is a 3 to 6 membered nitrogen-linked heterocyclic ring, wherein:
the nitrogen-linked heterocyclic ring optionally comprises one or two additional heteroatoms selected from nitrogen, oxygen or sulphur;
the nitrogen linked heterocyclic ring is optionally linked to a second 3 to 6 membered ring by a spiro carbon atom, the second 3 to 6 membered ring optionally comprising one or two further heteroatoms selected from nitrogen, oxygen or sulphur; and/or
R is optionally substituted with one or more substituents selected from H, oxo, (1-6C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, cyano, hydroxyl, amino, nitro, (1-4C)alkoxy, (1-4C)haloalkyl, (1-4C)haloalkoxy;
or a pharmaceutically acceptable salt or solvate thereof;
for the use in the treatment of HIV in patients carrying the HLA risk allele B*57:01.

2. A compound of the formula (I), shown below: 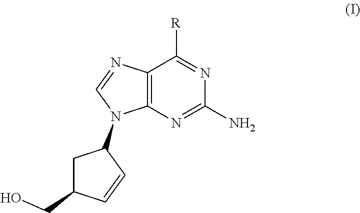
wherein R is a 3 to 6 membered nitrogen-linked heterocyclic ring, wherein:
the nitrogen-linked heterocyclic ring optionally comprises one or two additional heteroatoms selected from nitrogen, oxygen or sulphur;
the nitrogen linked heterocyclic ring is optionally linked to a second 3 to 6 membered ring by a spiro carbon atom, the second 3 to 6 membered ring optionally comprising one or two further heteroatoms selected from nitrogen, oxygen or sulphur; and/or
R is optionally substituted with one or more substituents selected from H, oxo, (1-6C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-4C)alkyl, halo, cyano, hydroxyl, amino, nitro, (1-4C)alkoxy, (1-4C)haloalkyl, (1-4C)haloalkoxy;
with the proviso that R is not an unsubstituted azetidin-1-yl ring;
or a pharmaceutically acceptable salt or solvate thereof.

3. A compound according to claim 1, or a pharmaceutically acceptable salt or solvate thereof, wherein R is a group of the formula: 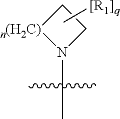
wherein: [symbol]
denotes the point of attachment;
R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
n is an integer selected from 1 to 3; and
q is an integer selected from 0 to 3.

4. A compound according to claim 1, wherein R is a group of the formula: 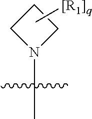
wherein: [symbol]
denotes the point of attachment;
R₁ is a substituent selected from oxo, (1-4C)alkyl, (3-6C)cycloalkyl, halo, cyano, hydroxyl, amino, nitro, (1-4C)alkoxy, (1-2C)haloalkyl, (1-2C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy; and
q is an integer selected from 1 to 3.

5. A compound according to claim 1, wherein R is a group of the formula: 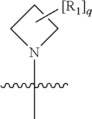
wherein: [symbol]
denotes the point of attachment;
R₁ is a substituent selected from oxo, (1-4C)alkyl, (3-6C)cycloalkyl, halo, cyano, hydroxyl, amino, (1-4C)alkoxy, (1-2C)haloalkyl, (1-2C)haloalkoxy, carboxyl, carbamoyl, sulphamoyl, amido, cycloalkyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl or cycloalkyl moiety is optionally further substituted with one or more substituents selected from (1-2C)alkyl, halo, cyano, hydroxyl, amino, (1-2C)alkoxy, (1-2C)haloalkyl, or (1-2C)haloalkoxy; and
q is an integer selected from 1 or 2.

6. A compound according to claim 1, wherein R is a group of the formula: 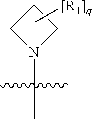
wherein: [symbol]
denotes the point of attachment;
R₁ is a substituent selected from oxo, (1-4C)alkyl, (3-6C)cycloalkyl, halo, cyano, hydroxyl, amino, (1-4C)alkoxy, (1-2C)haloalkyl, (1-2C)haloalkoxy, carboxyl, carbamoyl, sulphamoyl, amido, cycloalkyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl or cycloalkyl moiety is optionally further substituted with one or more substituents selected from (1-2C)alkyl, halo, cyano, hydroxyl, amino, (1-2C)alkoxy, (1-2C)haloalkyl, or (1-2C)haloalkoxy; and
q is an integer selected from 1 or 2.

7. A compound according to claim 1, wherein R is a group of the formula: 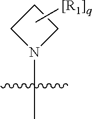
wherein: [symbol]
denotes the point of attachment;
R₁ is a substituent selected from (1-4C)alkyl, (3-4C)cycloalkyl, halo, cyano, hydroxyl, amino, (1-4C)alkoxy, (1-2C)haloalkyl, (1-2C)haloalkoxy, cycloalkyl-(1-2C)alkyl, wherein Rₐ and R_{b} are independently selected from H or (1-4C)alkyl, and wherein any alkyl or cycloalkyl moiety is optionally further substituted with one or more substituents selected from (1-2C)alkyl, halo, cyano, hydroxyl, amino, (1-2C)alkoxy, or (1-2C)haloalkyl; and
q is an integer selected from 1 or 2.

8. A compound according to claim 1, wherein R is a group of the formula: 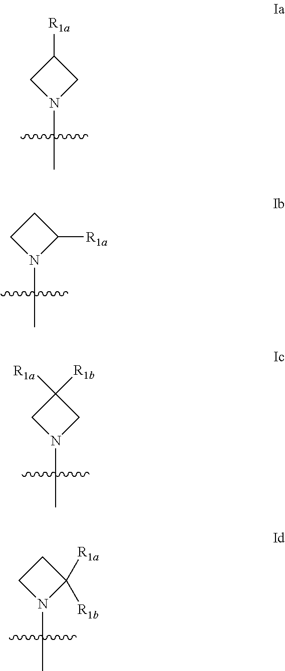
wherein:
R₁ₐ and R_{1b} are each a substituent group R₁ as defined in any one of claims 3 to 7.

9. A compound according to claim 8, wherein:
R1a a substituent selected from (1-4C)alkyl, (3-6C)cycloalkyl, halo, amino, cyano, hydroxy, amino, nitro, (1-4C)alkoxy, (1-2C) haloalkyl, (1-2C)haloalkoxy, carboxyl, carbamoyl, sulphamoyl or amido; and
R1b a substituent selected from (1-4C)alkyl, halo or hydroxy;
with the proviso that R1a and R1b cannot both be H.

10. A compound according to claim 8, wherein:
R1a is selected from (1-4C)alkyl, (3-6C)cycloalkyl, halo, amino, cyano, hydroxy, amino, nitro, (1-4C)alkoxy, (1-2C)haloalkyl or (1-2C)haloalkoxy; and
R1b is (1-2C)alkyl;
with the proviso that R1a and R1b cannot both be H.

11. A compound according to claim 2, which is selected from any one of the following: 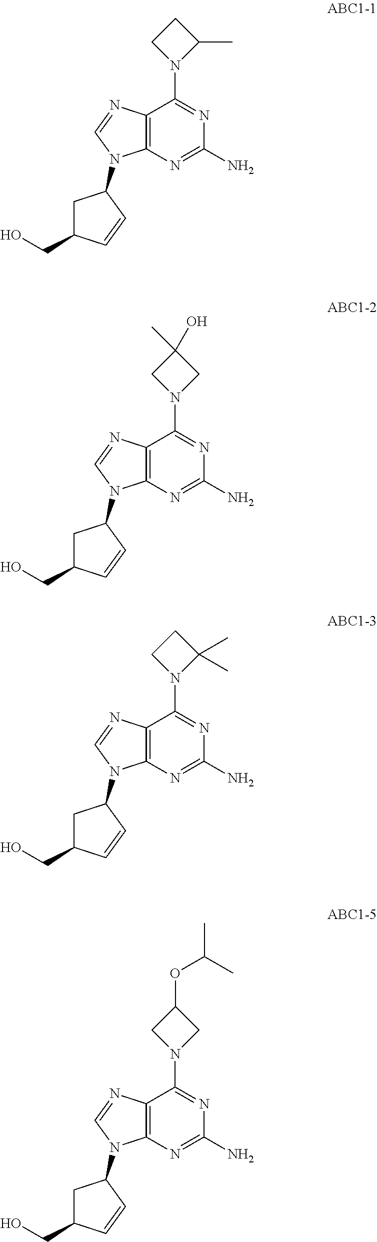 [SMILES: CC1CCN1c1nc(N)nc2c1ncn2[C@H]1C=C[C@@H](CO)C1 ; CC1(O)CN(c2nc(N)nc3c2ncn3[C@H]2C=C[C@@H](CO)C2)C1 ; CC1(C)CCN1c1nc(N)nc2c1ncn2[C@H]1C=C[C@@H](CO)C1 ; CC(C)OC1CN(c2nc(N)nc3c2ncn3[C@H]2C=C[C@@H](CO)C2)C1] 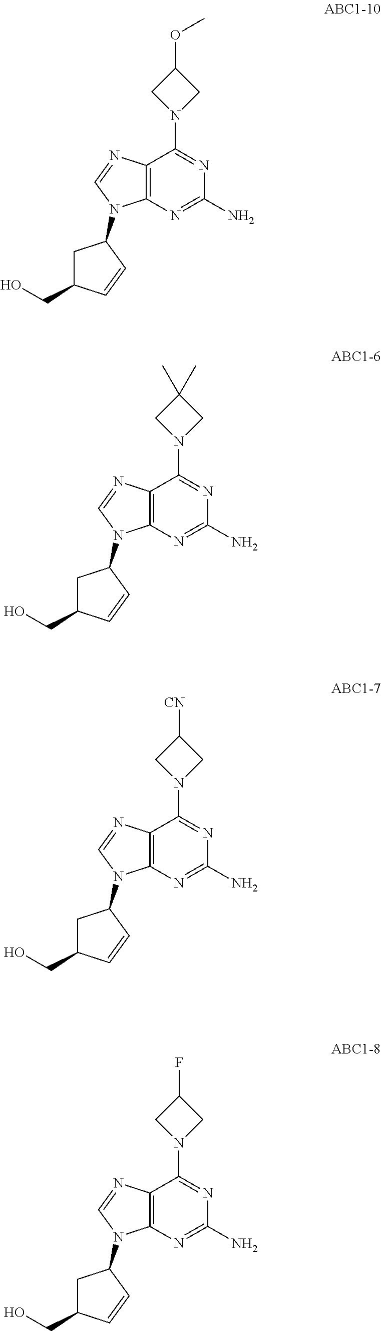 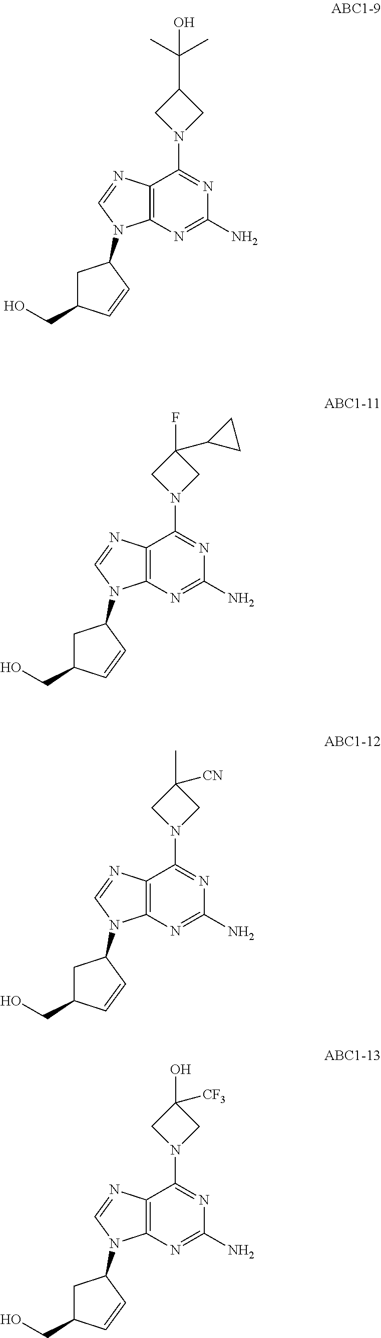 [SMILES: CC(C)(O)C1CN(c2nc(N)nc3c2ncn3[C@H]2C=C[C@@H](CO)C2)C1 ; Nc1nc(N2CC(F)(C3CC3)C2)c2ncn([C@H]3C=C[C@@H](CO)C3)c2n1 ; CC1(C#N)CN(c2nc(N)nc3c2ncn3[C@H]2C=C[C@@H](CO)C2)C1 ; Nc1nc(N2CC(O)(C(F)(F)F)C2)c2ncn([C@H]3C=C[C@@H](CO)C3)c2n1] 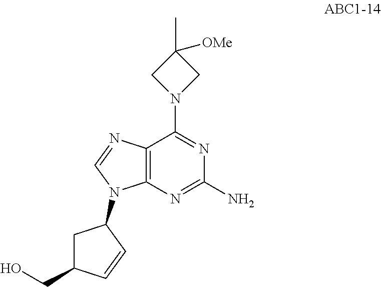 [SMILES: COC1(C)CN(c2nc(N)nc3c2ncn3[C@H]2C=C[C@@H](CO)C2)C1]
or a pharmaceutically acceptable salt or solvate thereof.

12. A compound according to claim 1, which is: 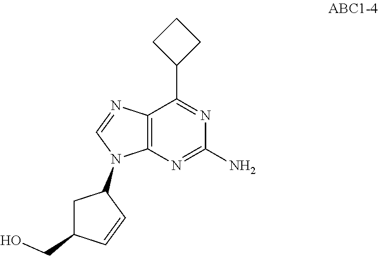
or a pharmaceutically acceptable salt or solvate thereof.

13.-16. (canceled)

17. A method of treating HIV in a patient in need of such treatment, said method comprising administering to said patient a therapeutically effective amount of a compound according to claim 2, or a pharmaceutically acceptable salt or solvate thereof.

18. The method of claim 17, wherein the HIV is HIV-1.

19. The method of claim 18, wherein the patient is carrying the HLA risk allele B*57:01.

20. The method of claim 17 further comprising the step of administering to the patient one or more additional antiviral agents.

21. A compound according to claim 2, or a pharmaceutically acceptable salt or solvate thereof, wherein R is a group of the formula: 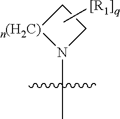
wherein: [symbol]
denotes the point of attachment;
R₁ is a substituent selected from oxo, (1-4C)alkyl, (3-6C)cycloalkyl, halo, cyano, hydroxyl, amino, nitro, (1-4C)alkoxy, (1-4C)haloalkyl, (1-4C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy;
n is an integer selected from 1 to 3; and
q is an integer selected from 0 to 3.

22. A compound according to claim 2, wherein R is a group of the formula: 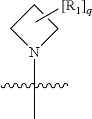
wherein: [symbol]
denotes the point of attachment;
R₁ is a substituent selected from oxo, (1-4C)alkyl, (3-6C)cycloalkyl, halo, cyano, hydroxyl, amino, nitro, (1-4C)alkoxy, (1-2C)haloalkyl, (1-2C)haloalkoxy, carboxyl, carbamoyl, sulphamoyl, amido, aryl, heterocyclyl, heteroaryl, aryl-(1-2C)alkyl, (3-6C)cycloalkyl-(1-2C)alkyl, heteroaryl-(1-2C)alkyl, heterocyclyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl, cycloalkyl, aryl, heterocyclyl, heteroaryl, aryl-alkyl, cycloalkyl-alkyl, heteroaryl-alkyl or heterocyclyl-alkyl group is optionally further substituted with one or more substituents selected from (1-2C)alkyl, halo, cyano, hydroxyl, amino, nitro, (1-2C)alkoxy, (1-2C)haloalkyl, or (1-2C)haloalkoxy; and
q is an integer selected from 1 to 3.

23. A compound according to according to claim 2, wherein R is a group of the formula: 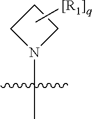
wherein: [symbol]
denotes the point of attachment;
R₁ is a substituent selected from oxo, (1-4C)alkyl, (3-6C)cycloalkyl, halo, cyano, hydroxyl, amino, (1-4C)alkoxy, (1-2C)haloalkyl, (1-2C)haloalkoxy, carboxyl, carbamoyl, sulphamoyl, amido, cycloalkyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl or cycloalkyl moiety is optionally further substituted with one or more substituents selected from (1-2C)alkyl, halo, cyano, hydroxyl, amino, (1-2C)alkoxy, (1-2C)haloalkyl, or (1-2C)haloalkoxy; and
q is an integer selected from 1 or 2.

24. A compound according to according to claim 2, wherein R is a group of the formula: 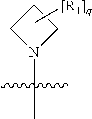
wherein: [symbol]
denotes the point of attachment;
R₁ is a substituent selected from oxo, (1-4C)alkyl, (3-6C)cycloalkyl, halo, cyano, hydroxyl, amino, (1-4C)alkoxy, (1-2C)haloalkyl, (1-2C)haloalkoxy, carboxyl, carbamoyl, sulphamoyl, amido, cycloalkyl-(1-2C)alkyl, C(O)Rₐ, C(O)ORₐ, OC(O)Rₐ, C(O)N(Rₐ)R_{b}, N(Rₐ)C(O)R_{b} or S(O)_{y}Rₐ, wherein y is 0, 1 or 2 and Rₐ and R_{b} are independently selected from H or (1-4C)alkyl, and wherein any alkyl or cycloalkyl moiety is optionally further substituted with one or more substituents selected from (1-2C)alkyl, halo, cyano, hydroxyl, amino, (1-2C)alkoxy, (1-2C)haloalkyl, or (1-2C)haloalkoxy; and
q is an integer selected from 1 or 2.
